(19) United States
(12) Patent Application Publication
(10) Pub. No.: US 2019/0234955 A1
(43) Pub. Date: Aug. 1, 2019

(54) EXOSOME-GUIDED TREATMENT OF CANCER

(71) Applicant: Nant Holdings IP, LLC, Culver City, CA (US)

(72) Inventor: Patrick Soon-Shiong, Culver City, CA (US)

(21) Appl. No.: 16/312,185

(22) PCT Filed: Jun. 21, 2017
(86) PCT No.: PCT/US2017/038515

§ 371 Date: Dec. 20, 2018

Related U.S. Application Data
(60) Provisional application No. 62/352,753, filed on Jun. 21, 2016.

(51) Int. Cl. G01N 33/68

(52) U.S. Cl. CPC G01N 33/6842; G01N 2800/52; G01N 2570/00

(57) ABSTRACT

Systems and methods of monitoring treatment of a patient use information gained from exosomes, wherein the treatment target that was identified from a tumor is followed in exosomes in a biological fluid outside the tumor.

This application claims priority to US provisional patent application with the Ser. No. 62/352753, filed Jun. 21, 2016.

### FIELD OF THE INVENTION

The field of the invention is monitoring treatment of cancer via exosomes, and especially via protein analysis of exosomes where the protein is associated with a mutation that is known to drive growth, metastasis, and/or proliferation.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Omics analysis has increasingly become a tool for determination of clinically relevant targets in the treatment of various diseases, and especially cancer. While omics analysis allows for critical insights into the diseased tissue and potential treatment options, monitoring treatment progression or success is typically not viable as such monitoring would require re-sampling the diseased tissue on a frequent basis. Alternatively, exosomes can be employed as a proxy to a biopsy in certain circumstances since cancer cells are known to shed exosomes in substantial quantities. For example, U.S. Pat. Nos. 8,021,847 and 8,476,017 teach use of exosomes as diagnostic tool to identify RNA sequences known to be associated with a disease. However, such approach fails to provide direct functional information of treatment and is less useful where the RNA sequence is also present in non-diseased tissue.

More recently, exosomes have also been reported to influence the biology of the tumor microenvironment (see e.g., Molecular Cancer 2016; 15:42, or Semin Cell Dev Biol 2015; 40:72-81) as well as immune responses (see e.g., Nat Rev Immunol 2014; 14(3):195-208). In addition, exosomes were reported to contain retrotransposon elements and amplified oncogene sequences (see e.g., Nat Commun 2011; 2:180).

Therefore, exosomes have also been proposed as therapeutic agents as is disclosed in, for example, US 2011/0053157. However, while tumor-derived exosomes have been shown to be potent anticancer vaccines in animal models, driving antigen-specific T and B cell responses, more recent literature concerning tumor derived exosomes strongly suggests the vesicles to play a significant immunosuppressive role (see e.g., Vaccines 2015, 3, 1019-1051). The '157 reference also teaches use of exosome associated RNA in the identification of potential treatment targets that can then be used to monitor treatment. Similarly, various exosome associated miRNAs were reported as potential markers (see e.g., Molecular Cancer (2016) 15:42).

Therefore, even though the field of exosomes has benefitted from considerable research efforts, reliable protein markers with a strong association in function, and especially a disease related function remained elusive. Thus, there is still a need for systems and methods that allow monitoring and validation of therapy using exosomes.

### SUMMARY OF THE INVENTION

The inventive subject matter is directed to various systems and methods of monitoring treatment of a patient using one or more patient- and disease-associated proteins that serve as targets in the treatment of the disease. Advantageously, such monitoring is highly specific to the disease and the patient, and provides direct information about the effect of the treatment, in particular where the treatment is an immune therapy targeting neoepitopes.

In one aspect of the inventive subject matter, the inventor contemplates a method of monitoring treatment of a patient that includes a step of using a plurality of omics data to identify a patient-specific disease-associated protein. In another step, presence and/or quantity of the patient-specific disease associated protein is determined in a first exosome, wherein the first exosome is obtained from a biological fluid of the patient prior to a treatment, and wherein the treatment targets the patient-specific disease associated protein. In yet another step, presence and/or quantity of the patient-specific disease associated protein is determined in a second exosome, wherein the second exosome is obtained from the biological fluid of the patient during or after the treatment. A patient record is then updated based on the determination of the at least one of presence and quantity of the patient-specific disease associated protein in the second exosome.

Most typically, the plurality of omics data are selected from the group consisting of whole genome sequencing data, exome sequencing data, transcriptome sequencing data, and proteome sequencing data, and/or the plurality of omics data include omics data from a diseased tissue and omics data from matched normal tissue.

In some embodiments, the patient-specific disease associated protein is identified using a pathway analysis algorithm (e.g., using PARADIGM to identify deregulated or rescue pathways) which will advantageously allow identification of non-mutated, silenced, underexpressed, or overexpressed genes. In other embodiments, the patient-specific disease associated protein is mutated or deregulated gene, which may identify cancer driver genes or genes involved in metastasis. Thus, contemplated patient-specific disease associated protein include a kinase, a receptor, a growth factor, a transcription factor, or a signal transduction-associated protein (e.g., where the treatment comprises a chemotherapy). In further embodiments, the patient-specific disease associated protein is a patient and tumor-specific neoepitope (e.g., where the treatment comprises an immune therapy). Additionally, it is contemplated that presence and/or quantity of the patient-specific disease associated protein may be determined using mass spectroscopic reaction monitoring (e.g., selected reaction monitoring, consecutive reaction monitoring, multiple reaction monitoring, or parallel reaction monitoring). Moreover, and if desired, contemplated methods may also include a step of analyzing a nucleic acid present in the first and/or second exosome, or a circulating tumor nucleic acid (e.g., ctRNA).

Exosomes may be isolated using non-specific entrapment and/or antibody-mediated capture, and biological fluids typically include whole blood, serum, plasma, and urine. Moreover, it is contemplated that the step of determining presence and/or quantity of the patient-specific disease associated protein in the second exosome may be repeated at least once, and that the step of updating the patient record will include a recommendation to modify the treatment.

Therefore, the inventor also contemplates a method of selecting an exosomal marker for monitoring treatment. Such method will preferably include a step of using a plurality of omics data to identify a patient-specific disease associated protein, and a further step of identifying a treatment composition targeting the patient-specific disease associated protein. At least one of presence and quantity of the patient-specific disease associated protein are determined in an exosome, wherein the exosome is obtained from a biological fluid of the patient prior to a treatment. The patient-specific disease associated protein is then selected for monitoring treatment upon determination that the disease associated protein is present in an amount sufficient for quantification (e.g., is at least an attomol).

Most typically, the of omics data are selected from the group consisting of whole genome sequencing data, exome sequencing data, transcriptome sequencing data, and proteome sequencing data, and/or the patient-specific disease associated protein is identified using a pathway analysis algorithm (e.g., using PARADIGM). While not limiting to the inventive subject matter, it is generally preferred that the omics data include omics data from a diseased tissue and omics data from matched normal tissue, and that the disease is a cancer.

With respect to the patient-specific disease associated protein it is contemplated that the protein may be an overexpressed protein or a mutated protein (e.g., a kinase, a receptor, a growth factor, a transcription factor, or a signal transduction-associated protein) that could be targeted with chemotherapy, and/or that the patient-specific disease associated protein may be a patient and tumor-specific neoepitope that could be targeted with immune therapy. It is still further contemplated that the presence and/or quantity of the patient-specific disease associated protein is determined using mass spectroscopic reaction monitoring.

In view of the above, the inventor also contemplates a method of monitoring immune therapy treatment of a patient. Preferred methods will include a step of determining presence and/or quantity of a patient- and tumor-specific neoepitope in a first exosome, wherein the first exosome is obtained from a biological fluid of the patient prior to a treatment, and wherein the immune therapy treatment targets the patient- and tumor-specific neoepitope. In another step, presence and/or quantity of the patient- and tumor-specific neoepitope are determined in a second exosome, wherein the second exosome is obtained from the biological fluid of the patient during or after the treatment. Most preferably, the steps of determining are performed using mass spectroscopic reaction monitoring (e.g., selected reaction monitoring, consecutive reaction monitoring, multiple reaction monitoring, and parallel reaction monitoring).

For example, suitable immune therapy treatments may include administration of a recombinant entity (e.g., adenovirus that is optionally replication deficient, irradiated bacterium, or an irradiated yeast) that comprises a nucleic acid encoding the patient- and tumor-specific neoepitope. Additionally, contemplated methods may include a step of analyzing a nucleic acid present in at least one of the first and second exosome, and/or a step of analyzing circulating tumor RNA in the biological fluid. Where desired, the immune therapy treatment may further comprise administration of a checkpoint inhibitor and/or an immune stimulatory cytokine.

Various objects, features, aspects and advantages of the inventive subject matter will become more apparent from the following detailed description of preferred embodiments.

### DETAILED DESCRIPTION

The inventor has discovered that various treatments of a patient, and especially cancer treatment, may be monitored by the detection and/or quantification of one or more exosomal proteins that are patient-specific and associated with the disease of the patient. In especially preferred methods, the proteins are qualitatively or quantitatively determined and may be on and/or in an exosome that is isolated from a bodily fluid of the patient. Moreover, the proteins are preferably the target of the treatment and will therefore provide direct and specific insight into the treatment efficacy. It should also be recognized that contemplated methods will allow following the treatment effects in a patient in real-time or near real-time.

As used herein, the term “patient” is interchangeable with the terms “subject” and “individual”, and refers to all animals shown to or expected to have exosomes. For example, the patient may be a mammal, a human or nonhuman primate, a dog, a cat, a horse, a cow, other farm animals, or a rodent.

In one exemplary aspect of the inventive subject matter, a patient diagnosed with a cancer may be subjected to a tumor biopsy in which a portion of the tumor used for omics analyses, typically using whole genome sequencing, transcriptome sequencing, and/or proteomics analysis. Preferably, the whole genome sequencing data are used in conjunction with whole genome sequencing data from matched normal tissue (i.e., healthy tissue from the same patient, such as blood or a healthy tissue portion of organ affected by tumor) to thereby identify cancer-associated changes that are also specific to the patient. While numerous algorithms for such comparative analysis are well known in the art, it is especially preferred that such analysis is done using synchronous incremental alignment of data files that are organized on the basis of positional reference information (e.g., BAM format, GAR format, etc.). For example, suitable algorithms include those in described in US 2012/0059670 and US 2012/0066001. In addition, it is generally preferred that the omics data (along with transcriptomics and proteomics data) are also used in a pathway analysis algorithm to identify potentially druggable targets or target pathways, or to identify one or more treatments that may restore sensitivity of the tumor to a drug. Among other suitable pathway analytic tools, especially contemplated pathway analysis algorithms are taught in WO 2011/139345, WO 2013/062505, and WO 2014/193982.

As should be readily appreciated, once suitable targets are identified on the basis of pathway analyses and/or mutational analysis, the patient may be treated with one or more chemotherapeutic agents that target the druggable target or target the drug sensitive pathway. Viewed from a different perspective, it should be recognized that so identified druggable targets and/or pathways provide patient-specific and disease associated proteins that are then used in treatment of the cancer. Similarly, patient-specific and disease associated proteins may be identified using pathway algorithms on the basis of expression level and/or mutational status (that, for example, results in over-activity or loss of activity). Alternatively, or additionally, omics analysis may also reveal the presence of one or more neoepitopes that are suitable for treatment with a cancer vaccine (e.g., via recombinant bacteria, yeast, or virus carrying a recombinant nucleic acid encoding the neoepitope in an expressible and MHC-presentable form). Therefore, patient-specific and disease associated proteins also include one or more patient and tumor specific neoepitopes.

It should be particularly appreciated that the patient-specific and disease associated proteins are established prior to start of the treatment (or a new round of treatment where prior treatment was ineffective) and that the identification of the disease associated proteins directly guides the type of effective treatment. Moreover, a biological fluid from the patient is obtained prior to the start of the treatment (or a new round of treatment where prior treatment was ineffective), and the presence and/or quantity of the patient-specific disease associated protein is determined in the exosomes in the biological fluid of the patient. By ascertaining a treatment for chemo and/or immunotherapy and by ascertaining presence of the target of the chemo and/or immunotherapy, treatment modalities are selected that not only are expected to have a higher likelihood of success, but that are also directly detectable and quantifiable during and after the course of chemo and/or immunotherapy. Therefore, at a later time during or after treatment, exosomes can be isolated from the patient and presence and/or quantity of the disease associated protein is determined to follow dynamic changes of the disease associated protein in real-time or near real-time.

In this context, it should be appreciated that tumor cells shed substantial quantities of exosomes, and that the changes in the tumor cell are directly reflected by the corresponding changes in the exosomes. Notably, the changes may be detectable on the surface of the exosomes (where they will typically be proteins) and/or in the lumen of the exosomes (where they may be siRNA, miRNA, mRNA, DNA, double minute chromosomes, proteins, metabolites, etc.). Moreover, and particularly where the target protein is present in only relatively small quantities, exosomal target identification and/or quantification will allow for an amplified signal that can be concentrated in a relatively fast manner (by concentration of the exosomes and/or exosomal proteins).

With respect to the plurality of omics data it is generally contemplated that the omics data are whole genome sequencing data, exome sequencing data, transcriptome sequencing data, and/or proteome sequencing data, and that the disease associated protein is preferably a neoepitope or identified using a pathway analysis algorithm (e.g., PARADIGM) where the disease associated protein is part of a signaling or signal transduction pathway. Most typically, the plurality of omics data will include omics data from the diseased tissue (tumor biopsy) and omics data from matched normal tissue (e.g., blood). While it is generally preferred that the disease is a cancer, it should be appreciated that numerous other diseases are also contemplated and particularly include inheritable diseases.

For example, and with respect to obtaining omics information from the patient to identify one or more neoepitopes it is generally contemplated that the omics data are obtained from one or more patient biopsy samples following standard tissue processing protocol and sequencing protocols. While not limiting to the inventive subject matter, it is typically preferred that the data are patient matched tumor data (e.g., tumor versus same patient normal), and that the data format is in SAM, BAM, GAR, or VCF format. However, non-matched or matched versus other reference (e.g., prior same patient normal or prior same patient tumor, or homo statisticus) are also deemed suitable for use herein. Therefore, the omics data may be ‘fresh’ omics data or omics data that were obtained from a prior procedure (or even different patient). For example, neoepitopes may be identified from a patient tumor in a first step by whole genome and/or exome analysis of a tumor biopsy (or lymph biopsy or biopsy of a metastatic site) and matched normal tissue (i.e., non-diseased tissue from the same patient such as peripheral blood) via location-guided synchronous comparison of the so obtained omics information.

Among other options, it is contemplated that genomic analysis can be performed by any number of analytic methods, however, especially preferred analytic methods include WGS (whole genome sequencing) and exome sequencing of both tumor and matched normal sample using next generation sequencing such as massively parallel sequencing methods, ion torrent sequencing, pyrosequencing, etc. Likewise, it should be appreciated that computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Of course, alternative file formats for sequence analysis (e.g., SAM, GAR, FASTA, etc.) are also expressly contemplated herein.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing software instructions that causes a processor to execute the disclosed steps associated with implementations of computer-based algorithms, processes, methods, or other instructions. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Of course, it should be appreciated that downstream analysis may be performed on the so identified sequence differences to identify those that lead to a new peptide sequence based on the cancer and patient specific mutation. Neoepitopes may therefore be identified by considering the type (e.g., deletion, insertion, transversion, transition, translocation) and impact of the mutation (e.g., non-sense, missense, frame shift, etc.), and may as such serve as a content filter through which silent and other non-relevant (e.g., non-expressed) mutations are eliminated. Moreover, filtering for suitable neoepitopes may also include filtering steps to eliminate genes that are transcribed and/or translated below a threshold value (typically below matched normal transcription and/or translation value).

In another example, omics data may also be analyzed using pathway analysis algorithms to identify genes that are mutated, over-, or under-expressed (relative to matched normal) and so contribute or are even causative to the disease. While various pathway analysis algorithms are known in the art and deemed suitable for use herein, an especially preferred pathway analysis algorithms is PARADIGM, which is described in WO2011139345, WO2013062505, and WO/2014/059036, and systems and methods as described in WO 2017/033154.

Moreover, pathway analysis and pathway model modifications can also be used in silico to identify drug treatment options and/or simulate drug treatment targeting pathway elements that are a determinant of or associated with a treatment-relevant parameter (e.g., drug resistance and/or sensitivity to a particular treatment) of a condition, and especially a neoplastic disease. More specifically, identified pathway elements can be modulated or modified in silico using a pathway analysis system and method to test if a desired effect could be achieved. For example, where a pathway model for drug resistance identifies over-expression of a certain element as critical to development of a condition (e.g., drug resistance against a particular drug), expression level of that element could be reduced in silico to thereby test in the same pathway analysis system and method if reduction of that element in silico could potentially reverse the cell to drug sensitivity. Such approach is particularly valuable where multiple cell lines representing multiple possible tumor variants are already available. In such a case, pathway analysis can be performed for each of the cell lines to so obtain a collection of cell line-specific pathway models. Such collection is particularly useful for comparison with data obtained from a patient sample, as the data for patient sample can be analyzed within the same data space as the collection, which ultimately allows for identification of treatment targets for the patient. Among other advantages, contemplated systems and methods therefore allow analysis of patient data from a tumor sample to identify multi-drug treatment before the patient has actually undergone the drug treatment.

Therefore, and viewed from a different perspective, it is also contemplated that various omics data from diseased cells and/or tissue of a patient can be used in a computational approach to determine a sensitivity profile for the cells and/or tissue, wherein the profile is based on a priori identification of pathways and/or pathway elements in a variety of similarly diseased cells (e.g., breast cancer cells). Most preferably, the a priori identified pathway(s) and/or pathway element(s) are associated with the resistance and/or sensitivity to a particular pharmaceutical intervention and/or treatment regimen. Once the sensitivity profile is established, treatment can be directly predicted from the a priori identified pathway(s) and/or pathway element(s), or identified pathways and/or pathway elements can be modulated in silico using known pathway modeling system and methods to so help predict likely outcomes for the pharmaceutical intervention and/or treatment regimen. Suitable systems and methods for such approach are described in WO 2014/193982.

It should be recognized that the pathway models may be generated from a set of omics data, or may be obtained from previous determinations. Therefore, contemplated systems and methods may also include a storage module that is coupled to the omic processing module, wherein the storage module stores one or more previously determined pathway models. It should also be recognized that the stored pathway models may correspond to ‘normal’ tissue or diseased tissue. Where the pathway model is from a diseased tissue, it should also be appreciated that the diseased tissue may be of a particular sub-type that is characterized by a sub-trait (e.g., sub-type that is treatment-resistant to a particular drug, sub-type that is from metastatic tissue, etc.). It is also contemplated that the omic data may be provided via the interface in numerous manners. For example, the data may be provided in a single file, or in a collection of distinct files, which may be provided by a service provider, from a library of previously stored, or from a sequencing device or sequence analysis system. Thus, the learning engine may further comprise or may be coupled to a genomic database, a BAM server, or sequencing device.

Depending on the particular path, it should be noted that the nature of the pathway element will change considerably, and with that the nature of the regulatory parameter. In general, it should be noted, however, that the regulatory parameter will determine the flow of a signal through the path from the pathway element to a downstream element. For example, where the pathway element is or comprises a DNA sequence, contemplated regulatory parameters will be those cellular entities that affect transcription (or other role) of the DNA sequence. Thus, contemplated regulatory parameters for a DNA sequence include one or more transcription factors, transcription activators, RNA polymerase subunits, cis-regulatory elements, trans-regulatory elements, (de)acetylated histones, (de)methylated histones, and/or repressors. Likewise, where the pathway element is or comprises an RNA sequence, it is contemplated that suitable regulatory parameters include factors that affect translation (or other activity) of the RNA. Consequently, such regulatory parameters include initiation factors, translation factors, RNA binding proteins, ribosomal RNA and/or proteins, siRNA, and/or polyA binding proteins. In the same way, here the pathway element is or comprises a protein, all factors affecting activity of that protein are deemed suitable regulatory parameters and may therefore include other proteins (e.g., interacting with the protein to form activated complex or complex with differential activity), chemical modification (e.g., phosphorylation, acylation, proteolytic cleavage, etc.).

Therefore, and using the results from the omics analysis to identify neoepitopes and/or other disease associated proteins (e.g., receptor, kinase, phosphatase, transcription factor, etc.), the inventor also contemplate a method of selecting an exosomal marker. In such method, a plurality of omics data from a patient are used to identify one or more disease associated proteins, and a drug is identifies as targeting the disease associated protein (e.g., a kinase inhibitor, a cell signaling inhibitor, etc.) where the therapy is a chemotherapy. Likewise, where the therapy is an immune therapy, the plurality of omics data from a patient are used to identify one or more neoepitopes, cancer associated antigens, and/or cancer specific antigens. In yet another step, it is verified that the disease associated protein is present (e.g., in a specific quantity) in or on an exosome. Most typically, and as already discussed above, the exosome is obtained from a biological fluid of the patient prior to a treatment. As will be readily appreciated, one or more disease associated proteins can then be selected upon determination or confirmation that the disease associated protein is indeed present in an amount sufficient for quantification.

Viewed from a different perspective, it is therefore contemplated that all manners of biochemical and omics analysis are appropriate, and that suitable disease associated proteins include one or more metabolites, one or more membrane lipid components, membrane associated proteins, transmembrane proteins, and intracellular proteins, as well as various nucleic acids. Consequently, contemplated methods of identifying will vary greatly and include biochemical analysis of tumor tissue (e.g., to detect or quantify enzymatic activity), whole genome and/or exome sequencing (e.g., to detect neoepitopes, genetic rearrangements, etc.), transcriptome analysis (e.g., over-expression or lack of expression), and proteomics analysis (e.g., to detect post-translational modification, quantity of expressed protein, etc.).

For example, with respect to the disease associated protein, the protein may be an overexpressed or mutated protein (e.g., kinase, receptor, growth factor, transcription factor, or signal transduction-associated protein). Where desired, contemplated methods may also include a step of analyzing a nucleic acid that may be present in the first and/or second exosome. For example, suitable nucleic acids include double minute chromosomes and RNA as further described below.

Most typically, omics (genomic, transcriptomic, and/or proteomic) analysis may be performed using BAMBAM and/or PARADIGM from tissue and matched normal samples that will readily identify disease associated proteins, especially including neoepitopes, druggable pathway alterations (e.g., over-activity of signaling, or loss of sensitivity towards a drug), driver genes/mutations, and genes associated with metastasis. Treatment with an appropriate drug or immunological regimen will then result in the reduction of cells expressing the neoepitope, and by extension, in a reduction of exosomes bearing the neoepitopes. Likewise, treatment with a drug may reduce expression of a receptor on a cancer cell, and by extension, reduce the quantity of expressed receptors on the exosomes.

More specifically, and among other suitable targets, omics analysis (and in less preferred aspects gene panel or other genetic analysis) may be employed to identify whether or not driver mutations are present in the cancer, and/or whether or not genes associated with metastasis are activate or suppressed in the cancer. For example, contemplated driver gene mutations and driver mutations include TP53, PIK3CA, KRAS, BRAF, PTEN, MLL3, APC, MLL2, ARID1A, NF1, FAT1, ANK3, MACF1, AHNAK, LAMA2, CDKN2A, EGFR, VHL, PBRM1, FAT2IDH1, NRAS, ATRX, ATM, RB1, NOTCH1, ARID2, etc. Further methods and systems to identify suitable cancer drivers can be found in Nature Methods 2013, Vol. 10 No. 11, 1081-4, and further examples of contemplated driver genes and driver mutations are published by Integrative Onco Genomics (Intogen.org).

Similarly, there are numerous known genes that are associated with metastasis and it is contemplated that all such genes are deemed suitable for use herein. For example, contemplated genes include AKAP12 (PKA regulation), BRMS1 (Transcription regulation), Caspase 8 (Apoptosis), CDH1 (Cell adhesion), CDH11(Cell adhesion), CD44 (Hyaluronic acid receptor), CRSP3 (Transcription regulation), DCC (Cell adhesion), DLC1 (Rho-GTPase activation), DRG1 (Angiogenesis), GAS1 (Apoptosis), Gelsolin (Actin depolymerization), KAI1 (Apoptosis), KISS1/KISS1R (Tumor dormancy maintenance), KLF17 (Transcription regulation), LSD1 (Chromotin remodeling), MAP2K4 (MAPKK signaling), MKK4 (MAPK signaling), MAK7 (MAPK signaling), MicroRNA-335, 126 (Suppression of SOX4, MERTK, PTPRN2, TNC), Nm23 (MAPK signaling), PEBP1 (Raf kinase inhibition), RhoGDI2 (Rho signaling), RRM1 (PTEN upregulation), TXNIP (Redox regulation).

Moreover, omics analysis may also identify genes or sequences that are amplified. For example, primary tumor samples of colorectal cancer patients with liver metastasis showed gain of chromosomes 7p, 8q, 13q and 20q and loss of chromosomes 1p, 8p, 9p, 14q, 17p and 22q. Genes that are located in the regions of chromosomal loss include MAP2K4, LLGL1, FBLN1, ELAC2, ALDH3A2, ALDH3A1, SHMT1, ARSA, WNT7B, TNFRSF13B, UPK3A, TYMP, RASD1, PEMT and TOP3A, all of which potentially serve as metastasis suppressors.

Once the disease associated proteins and treatment are established, a biological fluid of the patient (e.g., plasma, serum, or urine) is obtained prior to treatment and exosomes are then isolated or enriched from the biological fluid using methods well known in the art (e.g., via non-specific entrapment and subsequent affinity purification). For example, exosomes are typically isolated from a bodily fluid of a patient. As used herein, the term “bodily fluid” refers to a sample of fluid isolated from anywhere in the body of the subject, preferably a peripheral location, including blood, plasma, serum, urine, sputum, spinal fluid, pleural fluid, lymph fluid, fluid of the respiratory, intestinal tract, tear fluid, saliva, breast milk, ascitic fluid, and tumor cyst fluid.

As already noted before, isolation of exosomes can be performed in numerous manners, including non-specific methods such as ultracentrifugation and entrapment into polymeric networks (e.g., using ExoQuick™, commercially available from System Biosciences, 2438 Embarcadero Way, Palo Alto, Calif. 94303), co-precipitation with GlcNAc-carbohydrates via exosomal Annexin A5, and immune precipitation or magnetic separation using exosome specific surface markers, including CD9, CD63, CD81. Of course, it should be appreciated that all isolation methods may be combined to further enhance purity of the exosomes (e.g., where subsequent protein analysis is employed). However, and especially where the analysis is based on nucleic acid analysis, exosome enrichment via entrapment only may be suitable. Once enriched or isolated, exosomes may then be subject to various analytic processes to determine presence and/or quantity of the disease associated proteins.

Other methods of isolating exosomes from a biological fluid include those using differential centrifugation, ultracentrifugation, anion exchange and/or gel permeation chromatography, nanomembrane ultrafiltration, microfluidics, etc. (see e.g., U.S. Pat. Nos. 6,899,863, 6,812,023, 7,198,923). In especially preferred methods, exosomes can be non-specifically isolated using polymeric compositions (e.g., ExoQuick® (commercially available proprietary polymer from System Biosciences, 2438 Embarcadero Way, Palo Alto, Calif. 94303)), precipitation solutions (e.g., Exosome Precipitation Solution™, proprietary solution commercially available from Macherey-Nagel Inc., 2850 Emrick Blvd., Bethlehem, Pa. 18020). Likewise, suitable centrifugation protocols are well known (see e.g., Methods Mol Biol. 2015; 1295:179-209; Scientific Reports 5, Article number: 17319 (2015)).

Moreover, exosomes can also be further enriched for those originating from a specific cell type, for example, lung, pancreas, stomach, intestine, bladder, kidney, ovary, testis, skin, colorectal, breast, prostate, brain, esophagus, liver, placenta, etc. As exosomes often carry surface molecules/antigens from their donor cells, surface molecules/antigens may be used to identify, isolate and/or enrich for exosomes from a specific donor cell type. That way, exosomes originating from distinct cell populations can be analyzed for their protein and/or nucleic acid content. For example, tumor (malignant and non-malignant) exosomes will carry tumor-associated or tumor specific surface antigens and may be detected, isolated and/or enriched via these antigens. For example, suitable antigens include epithelial-cell-adhesion-molecule (EpCAM), which is specific to exosomes from carcinomas of lung, colorectal, breast, prostate, head and neck, and hepatic origin, but not of hematological cell origin. In another example, the surface antigen is CD24, which is a glycoprotein specific to urine exosomes. In yet another example, the surface antigen may be CD70, carcinoembryonic antigen (CEA), EGFR, EGFRvIII, Fas ligand, TRAIL, transferrin receptor, HSP72, etc.

Additionally, tumor specific exosomes may also be isolated on the basis of neoepitopes that are specific to a particular tumor and patient, where identification of the neoepitope is performed via omics analysis as described above. Such exosomes can be isolated using antibodies (most typically synthetic antibodies) and other high affinity binders such as those identified by phage display, mRNA display, etc. An exemplary method of generating high affinity binders against neoepitopes is disclosed in WO 2016/172722

Moreover, isolation of exosomes from specific cell types can also be accomplished using antibodies, aptamers, aptamer analogs, or molecularly imprinted polymers specific for a desired surface antigen. In one embodiment, the surface antigen is specific for a cancer type. In another embodiment, the surface antigen is specific for a cell type which is not necessarily cancerous. One example of a method of exosome separation based on cell surface antigen is provided in U.S. Pat. No. 7,198,923. As described in, e.g., U.S. Pat. Nos. 5,840, 867, 5,582,981, and WO/2003/050290, aptamers and their analogs specifically bind surface molecules and can be used as a separation tool for retrieving cell type-specific exosomes. Molecularly imprinted polymers also specifically recognize surface molecules as described in, e.g., U.S. Pat. Nos. 6,525,154, 7,332,553, and 7,384,589 and are suitable for isolating cell type-specific exosomes.

Once exosomes are isolated from the biological fluid of the patient, protein and/or nucleic acid analysis can be performed. In this context, it should be appreciated that protein(s) may be located within the lumen of the exosome, bound to the membrane, or on the surface of the exosome (e.g., as an ectodomain of a transmembrane protein, or as a membrane associated protein). Therefore, it should be noted that the exosome may be lysed or otherwise treated using various chemical agents, and especially contemplated agents include one or more detergents, chaotropic agents. Likewise, exosomes may also be treated with proteases to release membrane bound proteins. Alternatively, or additionally, exosomes may also be subjected to a physical process (e.g., sonication, electroporation, etc.) to release or make accessible the disease associated proteins. On the other hand, exosomes may also be used for protein analysis without further treatment (e.g., where the disease associated protein is present at the surface of the exosome and detected or quantified with a detectable label).

Most typically, the presence and quantity of the disease associated protein is determined using mass spectroscopic reaction monitoring, and especially using selected reaction monitoring (SRM), consecutive reaction monitoring (CRM), multiple reaction monitoring (MRM), or parallel reaction monitoring (PRM). Alternatively, protein analysis on exosomes may be performed in various other manners, including western blot, ELISA tests, binding to magnetic beads for FACS or other optical analysis, and various mass spectroscopic techniques, and the quantity of available exosomes and the particular disease associated protein will at least in part dictate the type of analysis used.

As noted earlier, it is generally preferred that the disease associated protein is determined and quantified prior to a treatment (e.g., chemotherapy and/or immunotherapy). With respect to subsequent determinations of the disease associated proteins once treatment has commenced, it is contemplated that such determination can be done under any schedule suitable for following the disease associated proteins. For example, determination can be done in a regular fashion (e.g., once or twice every week or month), or following other parameters (e.g., 12 or 24 hours after administration of a drug targeting the disease associated protein, and/or as a complimentary test after ultrasound, radiological, or other tomographical procedure). Likewise, the disease associated proteins need not be fixed over the course of treatment, but may be varied depending on observed treatment effects, biopsy results, subsequent omics analysis, etc.

In further contemplated aspects, it may be beneficial or otherwise desirable to extract nucleic acids (DNA, RNA, siRNA, shRNA, miRNA, etc.) from exosomes. Nucleic acid molecules can be isolated from exosomes using any number of procedures, all of which are well-known in the art and the particular isolation procedure will depend on the particular biological sample and type of nucleic acid. For example, where the nucleic acid is an RNA, the RNA may be reverse-transcribed into complementary DNA before further amplification. Such reverse transcription may be performed alone or in combination with an amplification step. One example of a method combining reverse transcription and amplification steps is reverse transcription polymerase chain reaction (RT-PCR), which may be further modified to be quantitative as described in U.S. Pat. No. 5,639,606. Other examples include hybridization to capture oligonucleotide (northern/southern blot), especially where the nucleic acid sequence is known or suspected. Furthermore, analysis of the nucleic acids in the exosomes may be quantitative or qualitative. For quantitative analysis, the amounts (expression levels), either relative or absolute, of specific nucleic acids of interest within the exosomes can be measured with methods known in the art. For qualitative analysis, the species of specific nucleic acids of interest within the exosomes, whether wild type or variants, may also be identified with methods known in the art.

In addition, it is contemplated that the bodily fluid may also be analyzed for one or more of the following circulating nucleic acids: circulating free RNA (cfRNA), circulating tumor RNA (ctRNA), circulating free DNA (cfDNA), and circulating tumor DNA (ctDNA). Such analysis may beneficially provide additional information to exosomal protein analysis and can be performed form the same biological fluid.

For example, ctRNA can be employed as a sensitive, selective, and quantitative marker for diagnosis and monitoring of treatment in conjunction with exosomal protein analysis, and advantageously allows repeated and non-invasive sampling of a patient from the same biological fluid. In most typical aspects, the ctRNA is isolated from a whole blood that is processed under conditions that preserve cellular integrity (to avoid contamination with RNA from lysed or otherwise damages cells) and stabilize ctRNA and/or ctDNA. Once separated from the non-nucleic acid components, the circulating nucleic acids are then quantified, preferably using real time quantitative PCR (of course, other circulating nucleic acids as described above are also deemed suitable for use herein).

Most typically, the biological fluid is the same as the biological fluid from which the exosomes are isolated. However, independent sampling is also contemplated herein. Thus, appropriate fluids include saliva, ascites fluid, spinal fluid, urine, etc, which may be fresh or preserved/frozen. For example, for suitable analyses, specimens can be accepted as 10 ml of whole blood drawn into cell-free RNA BCT® tubes or cell-free DNA BCT® tubes containing RNA or DNA stabilizers, respectively. Advantageously, ctRNA is stable in whole blood in the cell-free RNA BCT tubes for seven days while ctDNA is stable in whole blood in the cell-free DNA BCT Tubes for fourteen days, allowing time for shipping of patient samples from world-wide locations without the degradation of ctRNA or ctDNA. Moreover, it is generally preferred that the ctRNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Of course, it should be recognized that numerous other collection modalities are also deemed appropriate, and that the ctRNA and/or ctDNA can be at least partially purified or adsorbed to a solid phase to so increase stability prior to further processing.

As will be readily appreciated, fractionation of plasma and extraction of ctDNA and ctRNA can be done in numerous manners. In one exemplary preferred aspect, whole blood in 10 mL tubes is centrifuged to fractionate plasma at 1600 rcf for 20 minutes. The so obtained plasma is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). ctDNA and ctRNA are extracted from 2mL of plasma using Qiagen reagents. The extraction protocol is preferably designed to remove potential contaminating blood cells, other impurities, and maintain stability of the nucleic acids during the extraction. All nucleic acids were kept in bar-coded matrix storage tubes, with DNA stored at −4° C. and RNA stored at −80° C. or reverse-transcribed to cDNA that is then stored at −4° C. Notably, so isolated ctRNA can be frozen prior to further processing.

Quantification of isolated ctRNA can be performed in numerous manners, however, expression of analytes is preferably measured by quantitative real-time PCR of ct-cDNA using primers specific for each gene. For example, amplification can be performed using an assay in a 10 μL reaction mix containing 2 μL cDNA, primers, and probe. (3-actin can be used as an internal control for the input level of ct-cDNA. A standard curve of samples with known concentrations of each analyte can be included in each PCR plate as well as positive and negative controls for each gene. Delta Ct (dCT) were calculated from the Ct value derived from quantitative PCR (qPCR) amplification for each analyte subtracted by the Ct value of β-actin for each individual patient's blood sample. Relative expression of patient specimens is calculated using a standard curve of delta Cts of serial dilutions of Universal Human Reference RNA set at a gene expression value of 10 (when the delta CTs were plotted against the log concentration of each analyte).

With respect to suitable target nucleic acids, it should be appreciated that appropriate targets include all genes that are relevant to a disease and/or treatment of a disease. For example, disease targets include one or more cancer associated genes, cancer specific genes, genes with patient and tumor-specific mutations (neoepitopes), cancer driver genes, and genes known to be overexpressed in cancer. Still further contemplated target nucleic acids include those that encode the disease associated protein. Thus, suitable targets include those that encode ‘functional’ proteins (e.g., enzymes, receptors, transcription factors, etc.) and those that encode ‘non-functional’ proteins (e.g., structural proteins, tubulin, etc.), as well as those that encode neoepitopes. Viewed from a different perspective, suitable targets also include targets that are specific to a diseased cell or organ (e.g., PCA3, PSA, etc.), or targets that are commonly found in cancer patients, including various mutations in KRAS (e.g., G12V, G12D, G12C, etc) or BRAF (e.g., V600E), neoepitopes, checkpoint inhibitor ligands (e.g., PD-L1), etc.

Still further suitable targets for detection and quantification of ctRNA in conjunction with detection and/or quantification of disease related protein from exosomes include RNAs encoding one or more of ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDC73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHA5, EPHA7, EPHB1, ERBB2, ERBB3, ERBB4, EREG, ERG, ERRFI1, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HNF1A, HRAS, HSD3B1, HSP90AA1, IDH1, IDH2, IDO, IGF1R, IGF2, IKBKE, IKZF1, IL7R, INHBA, INPP4B, IRF2, IRF4, IRS2, JAK1, JAK2, JAK3, JUN, MYST3, KDM5A, KDM5C, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRSS8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, CD26, CD49F, CD44, CD49F, CD13, CD15, CD29, CD151, CD138, CD166, CD133, CD45, CD90, CD24, CD44, CD38, CD47, CD96, CD 45, CD90, ABCB5, ABCG2, ALCAM, ALPHA-FETOPROTEIN, DLL1, DLL3, DLL4, ENDOGLIN, GJA1, OVASTACIN, AMACR, NESTIN, STRO-1, MICL, ALDH, BMI-1, GLI-2, CXCR1, CXCR2, CX3CR1, CX3CL1, CXCR4, PON1, TROP1, LGRS, MSI-1, C-MAF, TNFRSF7, TNFRSF16, SOX2, PODOPLANIN, L1CAM, HIF-2 ALPHA, TFRC, ERCC1, TUBB3, TOP1, TOP2A, TOP2B, ENOX2, TYMP, TYMS, FOLR1, GPNMB, PAPPA, GART, EBNA1, EBNA2, LMP1, BAGE, BAGE2, BCMA, C10ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCRS, CCR6, CCR7, CCR8, CCR9, CCR10, CXCL1, CXCL2, CXCL3, CXCL5, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAGS, SPAG6, SPAG7, SPAG8, SPAG9, SPAG11A, SPAG11B, SPAG16, SPAG17, VTCN1, XAGE1D, XAGE2, XAGE3, XAGES, XCL1, XCL2, and XCR1. Of course, it should be appreciated that the above genes may be wild type or mutated versions, including missense or nonsense mutations, insertions, deletions, fusions, and/or translocations, all of which may or may not cause formation of a neoepitope in a protein expressed from such RNA. Such identified ctRNAs may also serve as a basis for selection of a treatment with a drug targeting the above noted gene products. In addition, combining quantitative or qualitative analyses of disease associated proteins with quantitative or qualitative analyses of ctRNA will provide not only insight into available treatments but also allows monitoring disease status and/or treatment effect from the same or a complementary vantage point. For example, while omics analysis from a biological fluid (e.g., blood) may identify a druggable target that can be followed by exosomal protein analysis, the same biological fluid may also provide information of the immune status, for example, via detection of PD-L1 ctRNA or information on other tumor specific markers.

It should be appreciated that by using disease associated proteins obtained from the exosomes, various advantages are realized. Among other things, where the disease associated protein is not a mutated protein and/or present in non-diseased cells, such proteins can still be quantified as cancer cells produce/release into the circulation significantly higher quantities of exosomes that healthy cells. Moreover, use of exosomes as claimed herein allows real-time (i.e., within hours or days post blood draw or isolation of biological fluid) detection of a treatment effect without need to obtain further tumor biopsies. In addition, intracellular proteins of tumor cells or otherwise diseased cells can be detected and quantified (by proxy via exosomes) without the need of a tumor biopsy. Such is especially beneficial where the disease associated proteins are detected form residual and/or circulating tumor cells that would otherwise not be visible or obtainable.

Particularly where the disease associated proteins are neoepitopes, it should be noted that detected/quantified neoepitopes will be directly correlated to the effect of immune therapy. Moreover, exosomal disease associated proteins may also be used to identify clonal populations, resistance, and/or susceptibility to checkpoint inhibition. In still further noted advantages, exosomal disease associated proteins can be monitored even in the absence of growth of the tumor. Thus, exosomal disease associated proteins are particularly suitable where the tumor is treatment resistant and/or has undergone other changes.

Consequently, it should be appreciated that the inventor contemplates in one aspect of the inventive subject matter, a method of monitoring ongoing treatment of a patient that is diagnosed with a cancer in which a plurality of omics data are used to first identify one or more disease associated proteins. Presence and/or quantity of the disease associated proteins are then determined in a first exosome obtained from a biological fluid of the patient prior to the treatment that targets the disease associated protein (e.g., chemotherapy to target a kinase, a receptor, or a receptor ligand, or immune therapy to target a tumor associated antigen, a tumor specific antigen, or a neoepitope, etc.). At a later time, the presence and/or quantity of the disease associated proteins are determined in a second exosome that is obtained from the biological fluid of the patient during or after the treatment. A patient record is then updated (e.g., to include a recommendation to modify the treatment) based on the determination of the presence and/or quantity of the disease associated protein in the second exosome.

Therefore, and viewed from a different perspective, the inventor also contemplates a method of selecting an exosomal marker. Especially preferred methods of selection include a step of using a plurality of omics data to identify one or more disease associated proteins, and identifying a drug (other other treatment) targeting the disease associated proteins. In still another step, presence and/or quantity of the disease associated protein are then determined in an exosome, wherein the exosome is obtained from a biological fluid of the patient prior to a treatment, and the disease associated protein is selected upon determination that the disease associated protein is present in an amount sufficient for quantification (e.g., an attomol of the disease associated protein where mass spectroscopy is employed).

Likewise, the inventor therefore also contemplates a method of monitoring treatment of a patient. Such method will preferably comprise a step of determining presence and/or quantity of one or more disease associated proteins in a first exosome that is obtained from a biological fluid of the patient prior to treatment (e.g., chemotherapy to target a kinase, a receptor, or a receptor ligand, or immune therapy to target a tumor associated antigen, a tumor specific antigen, or a neoepitope, etc.), and wherein the treatment targets the disease associated proteins, and another step of determining the presence and/or quantity of the disease associated protein in a second exosome, wherein the second exosome is obtained from the biological fluid of the patient during or after the treatment. Most typically, the steps of determining is performed using mass spectroscopic reaction monitoring.

In view of the above, it should therefore be appreciated that treatment of a patient can be monitored by determining presence and/or quantity of a disease associated protein in or on an exosome in a pre-treatment determination, where the exosomes are typically obtained from a biological fluid of the patient, and wherein the treatment targets the disease associated protein. During or after treatment, presence and/or quantity of the disease associated protein is once more determined in or on the exosome (which is yet again isolated from the biological fluid of the patient). Most preferably, determination of the disease associated protein is performed using mass spectroscopic reaction monitoring, and particularly selected reaction monitoring (SRM).

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Moreover, as used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of monitoring treatment of a patient, comprising:
using a plurality of omics data to identify a patient-specific disease-associated protein;
determining at least one of presence and quantity of the patient-specific disease associated protein in a first exosome, wherein the first exosome is obtained from a biological fluid of the patient prior to a treatment;
wherein the treatment targets the patient-specific disease associated protein;
determining at least one of presence and quantity of the patient-specific disease associated protein in a second exosome, wherein the second exosome is obtained from the biological fluid of the patient during or after the treatment; and
updating a patient record based on the determination of the at least one of presence and quantity of the patient-specific disease associated protein in the second exosome.

2-15. (canceled)

16. The method of claim 1 wherein the plurality of omics data include omics data from a diseased tissue and omics data from matched normal tissue.

17. The method of claim 1 wherein the patient-specific disease associated protein is identified using a pathway analysis algorithm.

18. (canceled)

19. (canceled)

20. The method of claim 1 wherein the patient-specific disease associated protein is a kinase, a receptor, a growth factor, a transcription factor, or a signal transduction-associated protein, and wherein the treatment comprises a chemotherapy targeting the kinase, the receptor, the growth factor, the transcription factor, or the signal transduction-associated protein.

21. The method of claim 1 wherein the patient-specific disease associated protein is a patient and tumor-specific neoepitope, and wherein the treatment comprises an immune therapy targeting the patient and tumor-specific neoepitope.

22. The method of claim 1 further comprising a step of analyzing a nucleic acid present in at least one of the first and second exosome, wherein the nucleic acid is a double minute chromosome.

23. The method of claim 1 wherein the presence and quantity of the patient-specific disease associated protein is determined using mass spectroscopic reaction monitoring.

24. The method of claim 23 wherein the mass spectroscopic reaction monitoring is selected from the group consisting of selected reaction monitoring, consecutive reaction monitoring, multiple reaction monitoring, and parallel reaction monitoring.

25. The method of claim 1 wherein the exosome is isolated using at least one of non-specific entrapment and antibody-mediated capture.

26-28. (canceled)

29. A method of selecting an exosomal marker for monitoring treatment, comprising:
using a plurality of omics data to identify a patient-specific disease associated protein, and identifying a treatment composition targeting the patient-specific disease associated protein;
determining at least one of presence and quantity of the patient-specific disease associated protein in an exosome, wherein the exosome is obtained from a biological fluid of the patient prior to a treatment;
selecting the patient-specific disease associated protein for monitoring treatment upon determination that the disease associated protein is present in an amount sufficient for quantification.

30-40. (canceled)

41. The method of claim 29 wherein the patient-specific disease associated protein is identified using a pathway analysis algorithm.

42-45. (canceled)

46. The method of claim 29 wherein the patient-specific disease associated protein is a kinase, a receptor, a growth factor, a transcription factor, or a signal transduction-associated protein, and wherein the treatment comprises a chemotherapy targeting the kinase, the receptor, the growth factor, the transcription factor, or the signal transduction-associated protein.

47. The method of claim 29 wherein the patient-specific disease associated protein is a patient and tumor-specific neoepitope, and wherein the treatment comprises an immune therapy targeting the patient and tumor-specific neoepitope.

48. The method of claim 29 wherein the presence and quantity of the patient-specific disease associated protein is determined using mass spectroscopic reaction monitoring.

49. The method of claim 29 wherein the amount sufficient for quantification is at least an attomol of the disease associated protein.

50. A method of monitoring immune therapy treatment of a patient, comprising:
determining at least one of presence and quantity of a patient- and tumor-specific neoepitope in a first exosome, wherein the first exosome is obtained from a biological fluid of the patient prior to a treatment, and wherein the treatment targets the patient- and tumor-specific neoepitope;
determining at least one of presence and quantity of the patient- and tumor-specific neoepitope in a second exosome, wherein the second exosome is obtained from the biological fluid of the patient during or after the treatment; and
wherein the steps of determining is performed using mass spectroscopic reaction monitoring.

51. The method of claim 50 wherein the immune therapy treatment comprises administration of a recombinant entity that comprises a nucleic acid encoding the patient- and tumor-specific neoepitope.

52. The method of claim 51 wherein the recombinant entity is at least one of an adenovirus that is replication deficient, an irradiated bacterium or an irradiated yeast.

53-56. (canceled)

57. The method of claim 50 further comprising a step of analyzing a nucleic acid present in at least one of the first and second exosome.

58. (canceled)

59. The method of claim 50 wherein the mass spectroscopic reaction monitoring is selected from the group consisting of selected reaction monitoring, consecutive reaction monitoring, multiple reaction monitoring, and parallel reaction monitoring.

60. (canceled)
